(19) United States
(12) Patent Application Publication
(10) Pub. No.: US 2021/0223262 A1
(43) Pub. Date: Jul. 22, 2021

(54) LYOSPHERE CRITICAL REAGENT KIT

(71) Applicants: Akhilesh BHAMBHANI, West Point, PA (US); Thorsten VERCH, West Point (US); Morrisa C. JONES, West Point, PA (US); Mary RETZLAFF, West Point, PA (US); Jessica R. SINACOLA, West Point, PA (US); Silikhone L. BOUARAPHAN, West Point, PA (US); Merck Sharp & Dohme Corp., Rahway, NJ (US)

(72) Inventors: Akhilesh Bhambhani, Doylestown, PA (US); Thorsten Verch, Blue Bell, PA (US); Morrisa C. Jones, Upper Darby, PA (US); Mary Retzlaff, Norristown, PA (US); Jessica R. Sinacola, Collegeville, PA (US); Silikhone L. Bouaraphan, Gilbertsville, PA (US)

(73) Assignee: Merck Sharp & Dohme Corp., Rahway, NJ (US)

(21) Appl. No.: 15/734,346

(22) PCT Filed: Jun. 3, 2019
(86) PCT No.: PCT/US2019/035103

§ 371 Date: Dec. 2, 2020

Related U.S. Application Data
(60) Provisional application No. 62/681,771, filed on Jun. 7, 2018.

(51) Int. Cl. G01N 33/68; G01N 33/58

(52) U.S. Cl. CPC G01N 33/6854; G01N 33/586; G01N 33/583

(57) ABSTRACT

The present invention provides lyophilized pellets of low concentrations of antibodies in the form of lyospheres. These lyospheres provide pre-measured antibody concentrations suitable for diagnostic and quality control assays.

### FIELD OF THE INVENTION

The present invention relates to single-use lyospheres containing a pre-measured antibody in an amount suitable for use in immunoassays. The invention also relates to methods and kits for using these lyospheres, particularly in immunoassays requiring multiple antibodies.

### BACKGROUND OF THE INVENTION

Antibodies are critical reagents of all immunoassays used for diagnostics, as well as in quality control for product release, stability and characterization. Industry standard practice generally involves storing antibody reagents as frozen liquids in multi-use vials. However, in general, liquid formulations are not an optimal solution due to issues with storage stability since the antibodies may aggregate and precipitate over time during storage especially when transported through different climatic zones or by improper storage (e.g. interruptions in the cool chain) and due to challenges with frozen reagents.

Although antibody reagents are often provided in a frozen form, lyophilization can be utilized to improve stability. Current technology for producing lyophilized formulations involves procedures such as dry blending, spray drying, and fluid bed drying. All of these procedures, however, have limitations that make them costly, inefficient or difficult to implement. For example, in dry blending technology, it is difficult to obtain homogeneous blends of reagents that have different densities. Moreover, homogeneity is particularly difficult to achieve when very small amounts of ingredients are mixed with large amounts of others. Once made homogeneous, it is extremely difficult to reproducibly (within 1 percent) dispense small amounts (less than about 10 mg) of the blended reagents.

Lyophilized antibody formulations have been described but often include cryoprotectants such as polymers, sugars, sugar alcohols, and albumins to overcome the problem with stabilization. See, e.g., International Patent Application Publication Nos. WO93/001835, WO93/000807, WO92/015331, WO89/011297, WO84/000890, Japanese Patent Application Publication No. JP60146833A; European Patent Application Publication Nos. EP 0303088 and EP0413188; U.S. Pat. No. 8,758,747; and Draber et al., 1995, J. Immun. Methods 181:37-43. Some cryoprotectants, such as albumins, do not lend themselves to single-use immunoassays as the cryoprotectants typically would need to be removed from the immunoassay due to the potential for cross-reacting with the antibody.

Current lyophilization approaches to antibody stabilization often rely on their lyophilization in a vial over an extended period of time, on the order of 3-5 days, and result in a dried cake (lyo cake). Typical lyo cakes can be hard to dissolve, thus potentially leading to concentration variability of the resuspended formulation. This problem can be exacerbated if parts of a broken lyo cake are stuck to the rubber stopper of the vial. In addition, traditional lyophilized antibody cakes are provided as multi-use formulations. This requires dilution prior to use in the assay, which can be cumbersome depending on the number of reagents needed.

Another challenge for lyophilizing antibodies is interfacial damage, especially at low antibody concentrations. See, e.g., Mehta et al., 2015, J Pharm Sci 104:1282-1290; Carpenter et al., 1999, Methods in Enzymology 309:236-255; and Cordes et al., 2012, J Pharm Sci 101:2307-2315.

Lyospheres or other frozen spheres containing reagents are described in U.S. Pat. No. 3,655,838 and International Patent Application Publication No. WO2001/004633, but have not been described for dilute antibody formulations.

What is needed are stable preparations of monoclonal antibodies that contain antibody in the small amounts suitable for single use.

### SUMMARY OF THE INVENTION

The present invention provides lyospheres in which a single lyosphere bead (or relatively few lyosphere beads) contains antibody in an amount which is suitable for single-use in an immunoassay. The lyospheres provide monoclonal antibody formulations that are highly stable and are suitable for use in diagnostic and quality control kits, particularly where multiple antibodies are used and the lyospheres can be color coded for ease of operation.

Specifically, the present invention provides a lyosphere comprising: a) an antibody in an amount from about 3 μg to about 45 μg; b) a buffer to provide a pH from about 6.0 to about 8.0; c) a surfactant; and d) one or more sugars. In certain embodiments, the buffer is histidine, succinate, phosphate, MOPS, or any combination thereof. In certain embodiments, the surfactant is a polysorbate or poloxamer. In one aspect of this embodiment, the surfactant is polysorbate 80, polysorbate 20 or poloxamer P188, which may be present at a concentration from about 0.015% to 0.1%. In certain embodiments, the sugars are selected from dextrose, sucrose, trehalose, lactose, raffinose, or any combination thereof. In certain aspects of this embodiment, the sugar is at a concentration of 7.5% or more.

In certain embodiments, the concentration of antibody is in a range from 100 g/ml to 500 μg/ml. In certain embodiments, the lyosphere is a substantially spherical discrete dried bead having a volume of from about 20 μl to about 100 μl. In certain embodiments, the ratio of mAb/sugar is in the range of from 1:3000 to 1:30000 (w/w).

In certain embodiments, the lyosphere further comprises an amino acid selected from arginine, proline, lysine, and leucine. In certain embodiments, the lyosphere further comprises hydropropylmethylcellulose (HPMC), which may be present at a concentration of about 1% to about 5%, e.g., at 2.5%. In certain embodiments, the lyosphere does not contain Bovine Serum Albumin (BSA).

In certain embodiments, the antibody is an IgG1, IgG3, or IgM antibody. In certain embodiments, the antibody is a capture antibody directed to a) a capsular polysaccharide from a S. pneumoniae serotype; b) a S. pneumoniae protein selected from PspA, PsaA, PhtA, PhtB, PhtD, or detoxified pneumolysin; or c) a carrier protein selected from CRM₁₉₇, diphtheria toxin, tetanus toxin, H. influenza protein D, or N. meningitides Outer Membrane Protein Complex (OMPC). In certain embodiments, the antibody is a detection antibody directed to a) a capsular polysaccharide from a S. pneumoniae serotype; b) a S. pneumoniae protein selected from PspA, PsaA, PhtA, PhtB, PhtD, or detoxified pneumolysin; or c) a carrier protein selected from CRM₁₉₇, diphtheria toxin, tetanus toxin, H. influenza protein D, or N. meningitides Outer Membrane Protein Complex (OMPC). In certain embodiments, the antibody is a reporter antibody selected from an AP-conjugate (alkaline phosphatase conjugated antibody) or HRP-conjugate (horse radish peroxidase conjugated antibody).

In certain embodiments, the lyosphere further comprises a colored dye. In certain embodiments, the lyosphere has a reconstitution time of less than 3 minutes or less than 2 minutes or less than 1 minute.

The present invention also provides a kit comprising two or more distinct lyospheres of the invention, wherein each of the two or more lyospheres comprise a capture antibody, a detection antibody, and/or a reporter antibody. In one embodiment, the lyospheres comprising the capture antibody, the lyospheres comprising the detection antibody and the lyospheres comprising the reporter antibody are each of a different color. In one aspect of this embodiment, each of the different lyospheres (comprising the capture antibody, detection antibody or reporter antibody) contains a dye of a different color. In another aspect of this embodiment, one of the lyospheres (comprising the capture antibody, detection antibody or reporter antibody) does not contain a dye and the remaining lyospheres contain a dye. Where there are two additional lyospheres, they contain dyes of a different color. In certain embodiments, the kit further comprises one or more containers containing one or more reconstitution liquids.

### BRIEF DESCRIPTION OF THE DRAWINGS

FIG. 1: Feasibility study with IgG1, IgG3, IgM, immune-sera, and enzyme-conjugated antibodies formulated as lyospheres (labeled “Lyobeads” in the figure) and as liquid solutions. Reagents were subjected to elevated temperature for different times and concentration-response curves were assessed by ELISA. Lyospheres were formulated in 12.5% Sucrose+12.5% Trehalose.

FIG. 2: Stability study comparing different lyosphere (labeled “Lyobeads” in the figure) formulations using 31 different antibody reagents directed against 15 different S. pneumoniae polysaccharide strains. Lyobeads were formulated in either 5% Sucrose+16% Trehalose or in 5% Lactose+16% Trehalose.

FIG. 3: Stability study with Lyospheres (labeled “Lyobeads” in the figure) formulated in 10% Raffinose using different antibody reagents.

### DETAILED DESCRIPTION OF THE INVENTION

The present invention is based, in part, on the surprising discovery that lyospheres can be prepared to include an antibody that provides the desired amount, at microgram quantities, to be used in a particular assay step. The amount of antibody present is several-fold lower than previously used in stable liquid or lyophilized formulations. Prior antibody lyosphere formulations contained ˜1 mg/ml antibody or greater, which was thought to be important to maintain protein stability and to reduce the fraction lost to adsorption to the storage container. See, e.g., International Patent Application Publication No. WO2014/093206.

Liquid antibody formulations are often lyophilized to provide storage stability. The lyophilization process involves freezing a liquid sample which is then subjected to a vacuum so that the ice in the frozen sample directly changes to water vapor or sublimes. After the removal of ice, the sample temperature is gradually increased (while still under vacuum) and water is desorbed from the remaining non-ice phase of the sample to form a lyo-cake. Lyophilization results in no water remaining in the lyo-cake.

In the lyospheres of the invention, the antibody concentrations are on the order of 500× less (˜0.25 mg/ml) than those found in previously described lyospheres (˜120 mg/ml). See, e.g., International Patent Application Publication No. WO2014/093206. At the concentrations used in the lyospheres of the invention, one of skill in the art would be genuinely concerned about the potential loss of antibody at the lyosphere surface. See, e.g., Xu et al., 2014, J Pharm Sci 103:1356-1366, describing increased degradation of protein when associated with the solid-air interface of lyophilized formulations. Notably, using the microgram antibody quantities described herein, a greater fraction of antibody would be associated with the solid-air interface and thus potentially subject to degradation.

One way to overcome this problem would be to use BSA as a filler to reduce the amount of inactivation and loss that occurs as a result of low-level binding to the storage vessel in dilute protein solutions (<1 mg/ml). See, e.g., Pierce Technical Resource TR0043.1, September 2005. However, BSA cannot be used as a component of lyospheres directly used in immunoassays (or in any case where BSA is retained) because the BSA would bind to the assay plate during the coating step and interfere with the immunoassay.

Importantly, the lyosphere formulation components selected for the lyospheres of the invention, including sugars used for stabilization, were found not to interfere with the binding of the capture antibody, antigen or secondary antibody to the plates. The single-use lyospheres contemplated herein, containing a pre-diluted antibody reagent, eliminate the risk of accidental cross-contamination between multiple reagents used in parallel, simplify the assay execution, and also provide an avenue to improve the stability of the reagents with optimization of the lyosphere formulation.

A further advantage of the lyospheres according to the invention is the faster freezing and drying of the beads due to improved surface area/volume ratio, which may further assist in avoiding damage to the antibodies during freezing. The lyospheres of the invention also exhibit no reduction in the antibody content and aggregate formation at a level that impacts the immunoassay, even after a long-term storage (e.g., 6 months) at 37° C. They are thus stable with regard to antibody content and purity. The improved surface area/volume ratio also allows faster reconstitution of the lyophilisates with water.

In certain embodiments, a lyosphere of the invention contains a monoclonal antibody (mAb) to a single target. In certain embodiments, a lyosphere may contain multiple antibodies directed to different targets in a single lyosphere. In certain embodiments, assay reagents such as buffers can be incorporated into separate lyospheres for ease of operation.

In certain embodiments, the lyosphere contains a ratio of mAb/sugar in a range from 1:3000 to 1:30000 on a weight by weight basis.

Besides the convenience of premeasured aliquots of antibodies, the lyospheres decrease sample variability due to user error. Analysts can use pre-diluted lyospheres (also referred to herein as lyobeads) without additional calculations, thereby simplifying assay procedures. As an additional benefit, the simplification also allows greater focus on critical steps. In addition, the lyospheres have an extended shelf life in a useful storage form, thereby maximizing the activity of the antibody and increasing its value as a component in a convenient kit format. The enhanced stability allows pre-diluting the antibodies for assay simplification and reduced waste of overhead volumes in storage tubes.

In addition, lyospheres facilitate the manufacture of diagnostic and assay kits containing the antibody in a stable, ready-to-use dilution. Contrary to liquid formulations, lyospheres improve kit stability, thus enabling the use of kits in low throughput scenarios where a kit lot may need to last for an extended period of time. Pre-diluted RTU (ready to use) reagents facilitate potential use of such kits with training limited to a protocol transfer.

As used herein, the term “antibody” may include antibodies modified for use in an immunoassay. This includes the addition of detectable moieties, including radioisotopes, fluorescent labels and enzyme-substrate labels. Antibodies may be from any species, including human, monkey, goat, rabbit, mouse, etc.

As used herein, the term “sugar” refers to any of a group of water-soluble carbohydrates of relatively low molecular weight. The term sugar includes reducing sugars (such as fructose and maltose), non-reducing sugars (such as sucrose and trehalose), sugar alcohols (such as xylitol and sorbitol) and sugar acids (such as gluconic acid and tartaric acid). In certain embodiments, a sugar refers to mono-, di-, tri-, and oligomeric sugar molecules comprising at most three, four, five or six monomeric sugar molecules.

As used herein with reference to the lyophilized pellets, i.e., lyospheres or lyobeads of the invention, the term “spherical” is intended to refer to substantially spherical pellets (e.g., greater than or equal to 50% spherical), and does not require that such pellets be perfectly spherical to fall within the scope of the present invention. The shapes of the pellets of the present invention will be substantially spherical based on their formation from droplets of solution suspended between a dispensing tip and a flat surface, in which the bulk of the surface area of the droplets is determined by surface tension. For example, a bead made on a flat plate may result in a semi-spherical shape (e.g., resembling half a soccer ball) whereas a bead made on a plate with a well may result in a shape more closely resembling a true sphere.

All ranges set forth herein are intended to be inclusive of the lower and upper limit of the range. All values set forth herein can vary by ±1%, ±2%, ±5%, ±10%, ±15%, or ±20%, the term “about” is also meant to encompass these variations.

### Preparation of Lyospheres

The general preparation of lyospheres is described in International patent Application Publication Nos. WO2013066769 and WO2015057541. Briefly, a method of making lyospheres of a biological material comprises loading an aliquot of a liquid composition comprising the biological material, such as an antibody, bispecific antibody, antibody-drug conjugate (ADC), etc., into a dispensing tip and dispensing the aliquot onto a solid, flat surface in such a way that the droplet remains intact while being dispensed. The term “solid, flat surface” means that there are no cavities or wells. Dispensing tips useful in the present invention include those with a round open end, and a pointed open end. Multiple dried pellets may be prepared simultaneously by loading simultaneously the desired number of aliquots of the liquid composition into a multichannel pipettor.

In one embodiment, the solid, flat surface is the top surface of a metal plate and is maintained at a temperature of −90° C. or lower. In some embodiments of the invention, the temperature of the metal plate is −150° C. or lower, or −180° C. or lower. In other embodiments, the temperature of the plate is within a range of about −90° C. to about −130° C., about −110° C. to about −150° C., about −150° C. to about −195° C. or −180° C. to about −196° C. The metal plate comprises a conductive, inert metal such as gold, silver, stainless steel, aluminum or copper. In a preferred embodiment, the metal plate is comprised of aluminum. In another preferred embodiment, the plate is stainless steel. In some embodiments, the metal plate is rectangular in shape, and in one preferred embodiment, the dimensions of the rectangular plate are 10 inches long×7 inches wide×0.4 inches thick.

The cold temperature of the metal plate is maintained by placing the bottom surface of the metal plate in physical contact with a heat sink. In one preferred embodiment, the heat sink comprises a plurality of fins composed of a temperature conductive metal. In some embodiments, the fins are spaced about 0.25 inches apart along the bottom surface of the metal plate, with each fin having a length of at least about one inch. For a 10 inch×7 inch plate, the heat sink preferably comprises thirty, one inch long fins.

The fins may be physically connected to the bottom of the metal plate using any of a multitude of approaches well-known in the art, for example, using metal screws, welding or gluing with a cryoglue. In such an embodiment, the term “bottom surface” means the surface of the plate that is physically connected to the plurality of fins. Alternatively, the metal plate and heat sink may be fabricated from a single metal block and in such a case, the skilled artisan will understand that the bottom surface of the metal plate and heat sink form part of the same functional feature and thereby in physical contact with each other.

The bottom surface of the metal plate rests on top of a metal reservoir containing a liquid cryogen such as liquid nitrogen. Other liquid cryogens that may be used in the heat sink include liquid propane, isopentane/hexane mixtures, argon and HFE-7100. The metal fins and reservoir are preferably made of the same conductive metal as used for the plate. Similar heat sinks may be purchased commercially, e.g., from M&M Metals, 1305W Crosby Road, Carrollton, Tex.

In another embodiment, the solid, flat surface is hydrophobic and is maintained above 0° C. during the dispensing step, and preferably between 4° C. and 25° C. The hydrophobic surface may comprise a chemically inert plastic such as polytetrafluoroethylene (PTFE), polypropylene and the like. The hydrophobic surface may be bonded to a different material or simply comprise the top surface of a thin film made using the hydrophobic material (e.g., PTFE, polypropylene). To freeze the liquid droplet, the film containing the dispensed droplet is chilled to a temperature that is below the freezing point of the liquid composition comprising the biological material, and preferably to a temperature of about 5° C. to 25° C. below the freezing point.

It is important to maintain the liquid droplet intact during the dispensing step. When the droplet is dispensed onto a cold metal surface (i.e., −90° C. or lower), one way of accomplishing this is to dispense the droplet at a dispensing speed and at a distance between top surface and the bottom of the dispensing tip (the “gap distance”) that prevents the droplet from freezing while any portion of the droplet is still in the tip, and maintains the dispensed droplet in simultaneous contact with the top surface of the metal plate and the bottom of the dispensing tip. This allows the droplet to freeze from the bottom up as it contacts the cold metal surface.

The dispensing speed and gap distance will depend upon the volume of the liquid droplet, and the shape of the open end of the dispensing tip, and may be readily determined experimentally. For a 250 μl lyobead, for example, this speed could range from 0.2 second to 3.0 seconds. Similarly for a 100 μl lyobead, for example, the dispensing speed could range from 0.1 second to 2 seconds. In preferred embodiments, the dispensing speed is within the range of about 3 ml/min to about 75 ml/min, about 5 ml/min to about 75 ml/min, about 3 ml/min to about 60 ml/min, about 20 ml/min to about 75 ml/min, or about 20 ml/min to about 60 ml/min, respectively. A suitable dispensing speed for preparing 50 and 20 microliter droplets is 4.5 ml/min of a composition with low solute concentration (5%) and 9 ml/min for a composition with high solute (25%) concentration.

In an alternative embodiment, the gap distance (i.e., between the open end of the dispensing tip and the top surface) is high enough so that the dispensed drop is in contact only with the top surface of the cold metal plate. To maintain the intactness and spherical shape of the droplet, the temperature of the metal surface is maintained well below −150° C. to ensure instantaneous freezing of the liquid droplet as it touches the surface. The gap distance will depend on the volume of the dispensed aliquot, but is usually at least 1 cm.

When the liquid droplet is dispensed onto a hydrophobic surface, the droplet is typically maintained intact in a substantially spherical shape by choosing a volume for the aliquot that will remain intact as the droplet touches the surface.

In certain embodiments, the dispensing tip or tips are connected to an automated dispensing unit capable of controlling the dispensing speed and the gap distance. Examples of automated dispensing units include the Biomek® FX Liquid Handling System and pipettors manufactured by Tecan.

After the pellets are frozen, they are placed in a lyophilization chamber and lyophilized. The steps of a typical lyophilization cycle useful in the present invention include loading, annealing, freezing, and one or more drying steps. In some embodiments, the drying step(s) is performed above 0° C. A preferred lyophilization cycle will keep the drying droplet below the collapse temperature and produce a dried pellet of substantially the same shape and size of the frozen droplet, and having a moisture content of about 0.1% to about 10%, about 0.1% to about 6%, about 0.1% to about 3% or 0.5% to about 5%. Examples of lyophilization cycles are shown below.

Lyophilization Parameters I

| | | Ramp Rate | | |
|---|---|---|---|---|
| Step | Temperature | (C./min) | Time | Pressure |
| Load: | −45° C. | 0.5 | 15 min | |
| Annealing: | −20° C. | 0.5 | 60 min | |
| Freezing: | −45° C. | 0.5 | 75 min | |
| Primary Drying: | 30° C. | 0.65 | 1350 min | 30 mTorr |
| Secondary | 30° C. | 0.65 | 270 min | 255 mTorr |
| Drying: | | | | |

Lyophilization Parameters II

| | | Ramp Rate | | |
|---|---|---|---|---|
| Step | Temperature | (C./min) | Time | Pressure |
| Load: | −45° C. | 0.5 | 15 min | |
| Annealing: | −20° C. | 0.5 | 60 min | |
| Freezing: | −45° C. | 0.5 | 75 min | |
| Primary Drying: | 15° C. | 0.65 | 1590 min | 30 mTorr |
| Secondary | 30° C. | 0.65 | 300 min | 255 mTorr |
| Drying: | | | | |

Lyophilization Parameters III

| | | Ramp Rate | | | |
|---|---|---|---|---|---|
| Step | Temperature | (C./min) | Time | | Pressure |
| Load: | −45° C. | 0.5 | 15 | min | |
| Annealing: | −20° C. | 0.5 | 60 | min | |
| Freezing: | −45° C. | 0.5 | 75 | min | |
| Primary Drying: | 15° C. | 0.65 | 28 | hr | 30 mTorr |
| Secondary | 15° C. | 0.65 | 5 | hr | 210 mTorr |

Drying:

The reconstitution time of the dried pellet can also be measured. The term “reconstitution time” refers to the time that is required to completely dissolve a lyosphere to produce a reconstituted liquid formulation that is clear. The lyophilized formulations can be reconstituted in preferably 15 minutes or less, 5 minutes or less, 2 minutes or 30 seconds or less.

After completion of lyophilization, the dried pellets may be placed in a container for bulk storage, or aliquoted into desired end-use container. Bulk storage containers include, e.g., plastic trays, metal trays, bottles, foil bags, and the like. The desired end-use container may be configured to receive a liquid for reconstitution directly in the container, e.g., a vial, or commercially available dual chamber containers, such as a dual-chamber cartridge pen device, dual chamber foil packet, a plastic tube with two or more chambers and designed to readily mix two or more components immediately before administration of the therapeutic or vaccine in the pellet. Alternatively, the end-use container may be adapted to allow removal of a desired number of pellets, e.g., such as a bead dispenser, and the removed pellets are then reconstituted with liquid in a separate container.

Alternative methods of lyophilizing biological materials in the form of spherically shaped pellets, i.e., beads, have also been described. In these methods, individual samples of the biological material are frozen and dried prior to placing a desired number of the dried samples into a storage container such as a glass vial. Historically, these methods relied on either (a) dispensing an aliquot of a liquid composition containing the desired amount of a biological material into a container of a cryogen such as liquid nitrogen, which results in direct contact of the biological material with the cryogen and/or (b) dispensing an aliquot of a liquid composition containing the biological material into a cavity present on a chilled solid plate, where the cavity contains the aliquot until it is frozen. It should also be noted that the use of plates with machined cavities often requires use of an automated system for detachment of the pellets from the cavity wall. Furthermore, reliance on a cavity to contain the liquid aliquot results in a volume restriction on the size of the aliquot and resulting pellet. Another approach, which is referred to as the die and punch method and uses a closed mold and compressive force to obtain a frozen pellet, suffers from a complex assembly design, leakage of fluid formation from the cavity and sticking of pellet to either the die or the punch. See, e.g., European Patent No. EP 0 617 613 B1.

### Pneumococcal Immunoassays

The lyospheres of the invention can be used in any assay which utilizes an antibody to detect the presence of or quantitate the amount of a pneumococcal antigen such as a capsular polysaccharide or a protein antigen.

To date, over 90 distinct pneumococcal serotypes have been identified based on their capsular polysaccharide composition. See, e.g., Song et al., 2013, J Korean Med Sci 28:4-15. A 23-valent pneumococcal vaccine, PNEUMOVAX 23, containing unconjugated capsular polysaccharides from serotypes 1, 2, 3, 4, 5, 6B, 7F, 8, 9N, 9F, 10A, 11A, 12F, 14, 15B, 17F, 18C, 19A, 19F, 20, 22F, 23F and 33F has been available since the 1980s. Because unconjugated capsular polysaccharides are poorly immunogenic in infants, a number of vaccines based on glyconjugates, where the capsular polysaccharide is conjugated to a carrier protein, have become available or are in development. A PCV-7 (PREVNAR), PCV-10 (SYNFLORIX) and PCV-13 (PREVNAR 13) are currently licensed for prevention of pneumococcal infections caused by the serotypes included in the vaccines in children. PCV-7 includes serotypes 4, 6B, 9V, 14, 18C, 19F, 23F. PCV-10 includes the serotypes in PCV-7+serotypes 1, 5, 7F, and 23F. PCV-13 includes the serotypes in PCV10+3, 6A, and 19A. Vaccines in development include PCV-15 which adds serotypes 22F and 33F.

Protein antigens include but are not limited to PcsB, PcpA, PhpA, PhtB, PhtD, PlyD1, PsaA, PspA, SP0148, SP1912, SP2108, StkP, and detoxified pneumolysin (dPly). See, e.g., Pichichero et al., 2015, Human Vaccines & Immunotherapeutics 12:194-205.

The lyospheres can also be used to quantitate the amount of a non-pneumococcal component of a pneumococcal conjugate vaccine such as a carrier protein. Carrier proteins used in licensed vaccines include but are not limited to CRM₁₉₇, diphtheria toxin, tetanus toxin, H. influenza protein D, and N. meningitides Outer Membrane Protein Complex (OMPC). See Pichichero, 2013, Hum Vaccin Immunother. 9:2505-2523.

Assays suitable for use with the lyospheres of the invention include, but are not limited to, competitive and non-competitive assay systems using techniques such as radioimmunoassays, enzyme immunoassays (EIA), enzyme linked immunosorbent assay (ELISA), sandwich immunoassays, precipitin reactions, gel diffusion reactions, immunodiffusion assays, agglutination assays, complement-fixation assays, immunoradiometric assays, fluorescent immunoassays, or immunoassays with alternative labels, protein A immunoassays, immunoelectrophoresis assays, and some label-free immunoassays such as nephelometry or plasmon surface resonance. For examples of immunoassay methods, see, e.g., U.S. Pat. Nos. 4,845,026 and 5,006,459.

The antibody may be labeled with a detectable moiety and used to detect the antigen in a sample (e.g., a patient's blood or a manufacturing lot). The antibody can be directly labeled or indirectly labeled (e.g., by a secondary or tertiary antibody that is labeled with a detectable moiety). Numerous labels are available including, but not limited to radioisotopes, fluorescent labels, and enzyme-substrate labels. Radioisotopes include, for example, ³⁵S, ¹⁴C, ¹²⁵I, ³H, and ¹³¹I. Fluorescent labels include, for example, rare earth chelates (europium chelates), fluorescein and its derivatives, rhodamine and its derivatives, dansyl, Lissamine, phycoerythrin and Texas Red. The labels can be conjugated to the antibody using the techniques disclosed in, for example, Current Protocols in Immunology, Volumes 1 and 2, Coligen et al., Ed., Wiley-Interscience, New York, N.Y., Pubs., (1991).

When using enzyme-substrate labels, the enzyme preferably catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes are described in O'Sullivan et al., Methods for the Preparation of Enzyme-Antibody Conjugates for use in Enzyme Immunoassay, in Methods in Enzymol. (Ed. J. Langone & H. Van Vunakis), Academic press, New York, 73: 147-166 (1981). Examples of enzyme-substrate combinations include, for example, horseradish peroxidase (HRPO or HRP) with hydrogen peroxidase as a substrate, alkaline phosphatase (AP) with para-Nitrophenyl phosphate as chromogenic substrate, and β-D-galactosidase (β-D-Gal) with a chromogenic substrate (e.g. p-nitrophenyl-β-D-galactosidase) or fluorogenic substrate 4-methylumbelliferyl-β-D-galactosidase.

In an ELISA, antibodies are used in conjunction to accurately quantify the antigen of interest. In a sandwich ELISA, a coating antibody or capture antibody is prepared, if not readily available from a commercial source, such that it is specific to an antigen and is anchored to a given surface. Additionally, a detection antibody is prepared which binds specifically to the antigen at a different epitope from the capture antibody. Lastly a reporter antibody (secondary/conjugate antibody) specific for the detection antibody is attached to a detectable reagent such as a radioactive, fluorescent or enzymatic reagent, for example horseradish peroxidase enzyme or alkaline phosphatase.

To carry out the ELISA, antibody specific to antigen is incubated on a solid support, e.g., a polystyrene dish that binds the antibody (capture or coating antibody). Any free protein binding sites on the dish are then covered by incubating with a non-specific protein such as bovine serum albumin. After a wash step to remove unbound material, the sample to be analyzed is incubated in the dish, during which time the antigen binds to the specific antibody attached to the polystyrene dish. Unbound sample is washed out with buffer.

In a standard ELISA, a reporter antibody specifically directed to the antigen is placed in the dish resulting in binding of the reporter antibody to any antibody bound to the antigen. The reporter antibody, or alternatively a secondary antibody, is linked to a detectable reagent such as horseradish peroxidase, alkaline phosphatase, or other labels. Unattached reporter antibody is then washed out. Reagents for label activity as needed e.g., a colorimetric or fluorogenic substrate, are then added to the dish. The label or substrate produces a detectable signal. The amount of signal developed in a given time period is proportional to the amount of antigen present in the sample. Quantitative results typically are obtained by comparing the sample signal to a standard curve signal.

In a sandwich ELISA, a detection antibody specifically directed to the antigen is added after capturing the antigen of interest. The detection antibody may be labeled directly or provide an anchor/binding site for a secondary (reporter) antibody linked to a detectable reagent such as horseradish peroxidase which may be simultaneously placed in the dish resulting in binding of the secondary to the detection antibody and the binding of the detection body to any antibody bound to the antigen. Wash steps are employed after each antibody addition.

The capture antibody can be a monoclonal antibody or polyclonal antibody or serum derived from different species including, but not limited to, human, llama, goat, chicken or mouse. In certain embodiments, the antibody is a mouse or human antibody against a capsular polysaccharide of a specific pneumococcal serotype. In another embodiment, the antibody is against a carrier protein such as CRM₁₉₇.

The concentration of the antibody (capture, detection or reporter) is between 0.5 to 5 μg/ml in the assay in an appropriate buffer. This concentration applies to the final concentrations in the assay (and is not what the concentration is in the lyosphere). Alternative lower or higher concentrations may be used in modified assay formats.

The detection antibody can be an antibody or polyclonal antibody or serum which in some way is different from the capture antibody, e.g., typically derived from a different species than the capture antibody including but not limited to human, llama, goat, chicken, or mouse. It is important that there is no interference between the two antibodies. Where an epitope is repetitive, the same antibody can be used for capture and detection. In certain embodiments, the antibody is a rabbit antibody against a capsular polysaccharide of a specific pneumococcal serotype. In another embodiment, the antibody is against a carrier protein such as CRM₁₉₇.

The secondary, label-conjugated antibody (reporter antibody) is specifically directed against the detection antibody and may be derived from different species, including but not limited to llama, goat, chicken, or mouse, or may be an anti-isotype antibody. The labeled secondary antibody is labeled directly wherein direct labels preferably include enzyme labels, fluorescent labels, radioactive labels or biotin. In certain embodiments, the secondary antibody is derived from donkey and conjugated to alkaline phosphatase as a label (resulting in an AP-Conjugate).

The enzymes used for labeling can either be a single enzyme, an oligomeric form of the enzyme, or an enzyme/antienzyme complex may be used. Preferably, the label can be an enzyme selected from alkaline phosphatase (AP), horseradish peroxidase (HRP), β-galactosidase and urease; a radioisotope selected from ¹²⁵I and ¹³¹I; a fluorescent label selected from a fluorochrome, FITC (fluorescein isothiocyanate) and TRITC (tetramethylrhodamine isothiocyante), or biotin.

In a particular embodiment, the invention is directed to lyospheres for use in an ELISA for a multivalent S. pneumoniae polysaccharide-protein conjugate vaccine.

In a particular embodiment, the invention is directed to lyospheres for use in an ELISA for a single S. pneumoniae polysaccharide-protein conjugate for inclusion in a multivalent vaccine. This is essential for the evaluation of antigen content and lot-to-lot consistency of manufacture of vaccines.

In an exemplary sandwich ELISA for a multivalent polysaccharide (Ps)-protein conjugate vaccine intended to measure total polysaccharide including both Ps conjugated to a carrier protein and free (i.e., unconjugated), Ps is captured and detected by serotype-specific antibodies and the Ps content is compared relative to a multivalent standard by parallel line analysis of dilution curves.

Serotype-specific capture monoclonal antibodies (mAb), either mouse or human, are coated directly on microtiter plates at 1-3 μg/ml. After a blocking step, dilution curves of standards and samples are applied across the coated microtiter plates. Immobilized polysaccharides are detected with a mixture of serotype-specific rabbit anti-PnPs (pneumococcal polysaccharide) detection mAbs and goat anti-rabbit Fc-specific antibody conjugated to alkaline phosphatase (AP Conjugate). A fluorescent signal is developed with 4-Methylumbelliferyl phosphate (4-MuP) (SuperPhos™, Virolabs).

The following are representative steps:

*At the steps marked with “*”, antibody reagents are prepared from prediluted lyobeads instead of traditional dilution from concentrated stock solutions. Lyobeads may contain, for example, 15 μg to 45 μg of antibody that is resuspended in 15 mL of buffer per bead for subsequent application to the plates. Thus, for a bead volume of 50 μL, the antibody concentration in each bead would range from 300 μg/mL to 900 μg/mL. Other antibody concentrations may be accommodated in lyobeads as applicable. Lyobeads for different antibody reagents may be colored by including dyes such as but not limited to commercially available blue or red food dyes at a concentration that does not interfere with the assay. Preferably, the detection antibody and the label-conjugated antibody beads will contain dyes to distinguish them. The capture antibody may contain, but preferably does not contain, dyes as they could interfere with antibody binding to the plate.

1. *Plate coating with coating/capture antibodies at an appropriate concentration/dilution, e.g. 1 μg/mL, in coating buffer (1 M ammonium sulfate, 10 mM Tris, pH 8.0, and 2000× diluted Diluent T). (Diluent T is 10 mM Tris, pH 7.7, 0.15 M NaCl, 0.05% v/v PS-20, and 1% (w/v) BSA).

2. Incubation at a temperature between 18 to 25° C. for about 90 minutes with shaking (˜600 rpm).

3. Washing of plates for 3× with 1× TBST wash buffer (Teknova, Hollister, Calif.).

4. Addition of Diluent T for blocking to reduce non-specific binding of subsequent samples and reagents.

5. Incubation at a temperature between 18 to 25° C. for a duration of about 15 mins up to 7 hours.

6. Washing of plates for 3× with 1× TBST wash buffer.

7. Add Diluent T and appropriately serially diluted samples and standards.

8. Incubation at a temperature between 18 to 25° C. for 90 minutes with shaking (˜600 rpm).

9. Washing of plates for 3× with 1× TBST wash buffer.

10. *Addition of diluted anti-Ps detection antibody and *AP-conjugated Ab in same Diluent T solution (Monoclonal Antibodies). Capture antibody beads were received in the form of 3 beads per vial, each containing 15 μg of antibody. These 3 beads were resuspended in 45 mL of water to a concentration of 1 μg/mL and used directly in the assay.

11. Incubation at a temperature between 18 to 25° C. for a duration of about 90 minutes.

12. Washing of plates for 6× with 1× TBST wash buffer.

13. Addition of 100 μL substrate (4-MuP) in all the wells.

14. Incubation at a temperature between 18 to 25° C. for a duration of 45 minutes±10 minutes.

15. Reading the OD at 450 nm.

In an exemplary sandwich ELISA for a multivalent polysaccharide-protein conjugate vaccine intended to measure the amount of conjugated polysaccharide (PnPs) present. Conjugated PnPS is captured with anti-CRM₁₉₇ monoclonal antibodies (mAbs), or antibodies to the appropriate carrier protein, and detected by serotype-specific anti-polysaccharide antibodies. The response is compared to a multivalent standard using parallel line analysis of the dilution curves. The conjugated polysaccharide content can be used as a drug product stability characterization test.

Anti-CRM₁₉₇ monoclonal antibodies (mAb) are coated directly on the microtiter plate. After a blocking step, dilution curves of standards and samples are applied across the coated microtiter plates. Immobilized polysaccharide conjugates are detected in a serotype-specific manner using rabbit anti-PnPS mAbs and a goat anti-Fc-specific rabbit antibody conjugated to alkaline phosphatase (AP Conjugate). A fluorescent signal is developed with 4-Methylumbelliferyl phosphate (4-MuP) (SuperPhos™, Virolabs).

Similarly to the ELISA described above, the following are representative steps. Steps marked with “*” use pre-diluted lyobeads to prepare reagents instead of traditional dilution from concentrated stock solutions:

1. Plate coating with 100 μl coating antibodies in coating buffer at an appropriate concentration (1 M ammonium sulfate, 10 mM Tris, pH 8.0) (e.g., Dilution is 1:5000 to 1:15000).

2. Incubation at a temperature between 18 to 25° C. for about 75 minutes±15 minutes with shaking (˜600 rpm).

3. Washing of plates for 3× with 250-300 μl/well 1× TBST wash buffer (Teknova, Hollister, Calif.).

4. Addition of 250 μl of Diluent T. (Diluent T is 10 mM Tris, pH 7.7, 0.15 M NaCl, 0.05% v/v PS-20, and 1% (w/v) BSA)

5. Incubation at a temperature between 18 to 25° C. for a duration of about 15 mins up to 2 hours.

6. Washing of plates for 3× with 250-300 μl 1× TBST wash buffer.

7. Add 100 μl Diluent T and appropriately serially diluted samples and standards.

8. Incubation at a temperature between 18 to 25° C. for 75 minutes±15 mins with shaking (˜600 rpm).

9. Washing of plates for 3× with 250-300 μl 1× TBST wash buffer.

10. *Addition of diluted anti-Ps detection antibody and *AP-conjugated Ab in same Diluent T solution.

11. Incubation at a temperature between 18 to 25° C. for a duration of about 75±15 minutes.

12. Washing of plates for 6× with 250-3001 μl 1× TBST wash buffer.

13. Addition of 100 μl substrate (4-MuP) in all the wells.

14. Incubation at a temperature between 18 to 25° C. for a duration of 50 minutes±10 minutes.

15. Reading the OD at 450 nm.

### Pneumococcal Lyospheres

The lyospheres of the invention contain an antibody useful to detecting the pneumococcal antigen of interest. Pneumococcal antigens of interest include capsular polysaccharides, pneumococcal proteins and unrelated carrier proteins. The lyospheres of the invention will generally also include stabilizers including one or more of sugars, buffer, surfactant, and optionally, amino acids and/or a gelling agent such as hydropropylmethylcellulose. In certain embodiments, the lyosphere does not contain BSA. The lyosphere can also contain a dye to differentiate between lyospheres containing the capture antibody, the detection antibody and, where present, the secondary label-conjugated (reporter) antibody to the detection antibody.

Antibodies suitable for use in the invention include antibodies to S. pneumoniae polysaccharides or proteins. Antibodies to carrier proteins used in pneumococcal conjugate vaccines can also be used. In certain embodiments, the antibody is directed to a S. pneumoniae capsular polysaccharide from a serotype selected from 1, 2, 3, 4, 5, 6A, 6B, 6C, 6D, 7B, 7C, 7F, 8, 9N, 9V, 10A, 11A, 12F, 14, 15A, 15B, 15C, 16F, 17F, 18C, 19A, 19F, 20, 21, 22A, 22F, 23A, 23B, 23F, 24F, 27, 28A, 31, 33F, 34, 35A, 35B, 35F, and 38. In certain embodiments, the antibody is directed to a carrier protein selected from CRM₁₉₇, diphtheria toxin, tetanus toxin, protein D, or pneumolysin (preferably detoxified).

The lyospheres of the invention will be produced with amounts of the antibody, for example, of up to 50 or 100 μg. In particular, antibody amounts from about 10 μg to 90 μg, or 15 μg to 90 μg. In general, the lyospheres have a volume of about 25 μl to 100 μl. The lyospheres of the invention will be produced with concentration ranges of the antibody, for example, of up to 1 mg/ml. Preferred concentration ranges are concentrations above 100 μg/ml. In particular, concentration ranges of 100 μg/ml to 900 μg/ml, or 300 μg/ml to 900 μg/ml.

Antibodies that are useful in this aspect of the invention can be of any isotype, including, but not limited to, the IgG1, IgG2, IgG2m4, IgG3, IgG4, or IgM. In specific embodiments, the antibody is an IgG1, IgG3, IgM isotype.

Suitable sugars for inclusion in the lyospheres include monosaccharides, disaccharides or trisaccharides. Representative monosaccharides include glucose, dextrose, mannose, galactose, fructose and sorbose. Representative disaccharides include sucrose, lactose, maltose or trehalose. A representative trisaccharide is raffinose. In certain embodiments, the sugars are selected from dextrose, sucrose, trehalose, lactose, raffinose, or any combination thereof.

Sugar alcohols, such as sorbitol, mannitol, inositol or xylitol, can be used instead of or in addition to a sugar. Mixtures of two or more different of the aforementioned sugar or sugar alcohols can also be used, such as mixtures of sucrose and trehalose.

The total amount of the sugar in the aqueous composition is typically greater than 7.5% and may range from 7.5-55% w/w, 15-50% w/w, 20-45% w/w, 25-45% w/w, 25-47.5% w/w, 25-40% w/w, 30-47.5% w/w, 30-40% w/w, 25-35% w/w or 27-30% w/w. When mono- and di-saccharides are employed, the amount of sugar is generally higher than 25% w/w, typically around 27-40% w/w. When trisaccharides are employed, the amount of sugar can be reduced to 10-15% w/w.

A buffer is included to maintain the pH within a desired range upon addition of acid, base, inorganic compound, organic compound or other solvent or diluent. What is meant by having a buffer to provide a specified pH range is that the formulation has that pH range prior to drying or after reconstitution. Representative buffers for use herein include, but are not limited to potassium phosphate, sodium phosphate, sodium acetate, histidine, HEPES, MOPS, Tris, Bis-Tris, imidazole, sodium citrate, sodium succinate, ammonium bicarbonate, and a carbonate. In certain embodiments, the buffer is histidine, succinate, phosphate, MOPS, or any combination thereof. The buffer may comprise a pH ranging from about pH 4 to about pH 10, a pH ranging from about pH 6 to about pH 8, and also, a pH of about pH 6 to about pH 7.

The buffers used should be present in a concentration of about 5 mM to about 100 mM. In some embodiments, the buffer concentration is in the range of about 10 mM to about 75 mM. In alternative embodiments, the concentration of the buffer is in the range of about 25 mM to about 75 mM, about 25 mM to about 100 mM, about 50 mM to about 100 mM, or about 50 mM to about 75 mM.

Suitable surfactants include all surfactants that are usually used in immunoassays. Surfactants may be added to reduce and/or prevent aggregation or to prevent and/or inhibit protein damage during processing conditions such as purification, filtration, freeze-drying, transportation, storage, and delivery. Surfactants that are useful in the formulations of the invention include, but are not limited to: nonionic surfactants such as polyoxyethylene sorbitan fatty acid esters (polysorbates, sold under the trade name Tween® (Uniquema Americas LLC, Wilmington, Del.)) including polysorbate-20 (polyoxyethylene sorbitan monolaurate), polysorbate-40 (polyoxyethylene sorbitan monopalmitate), polysorbate-60 (polyoxyethylene sorbitan monostearate), and polysorbate-80 (polyoxyethylene sorbitan monooleate); polyoxyethylene alkyl ethers such as Brij® 58 (Uniquema Americas LLC, Wilmington, Del.) and Brij® 35; poloxamers (e.g., poloxamer 188); Triton® X-100 (Union Carbide Corp., Houston, Tex.) and Triton® X-114; NP40; Span 20, Span 40, Span 60, Span 65, Span 80 and Span 85; copolymers of ethylene and propylene glycol (e.g., the Pluronic® series of nonionic surfactants such as Pluronic® F68, Pluronic® 10R5, Pluronic® F108, Pluronic® F127, Pluronic® F38, Pluronic® L44, Pluronic® L62 (BASF Corp., Ludwigshafen, Germany); and sodium dodecyl sulfate (SDS). Cationic surfactants may also be utilized in the formulations of the invention. Examples of cationic surfactants useful in the invention include, but are not limited to: benzalkonium chloride (BAK), benzethonium chloride, cetramide, cetylpyridinium chloride (CPC), and cetyl trimethylammonium chloride (CTAC), primary amines, secondary amines, tertiary amines, and quaternary amine salts.

In exemplary embodiments of the invention, the surfactant is a nonionic surfactant selected from the group consisting of: polysorbate 20, polysorbate 80, poloxamer P188, Brij®35, Pluronic® F-68 and Triton®. In some preferred embodiments, the surfactant is polysorbate 20, polysorbate 80 or poloxamer P188.

The amount of surfactant to be included in the formulations of the invention is an amount sufficient to perform the desired function, i.e., a minimal amount necessary to prevent protein aggregation, to prevent or inhibit the formation of particulates, to reduce the amount of aggregation of the lyophilized protein or antibody after reconstitution to an acceptable level, to allow ease of reconstitution or to provide a stability advantage during shipping and/or processing. Typically, the surfactant is present in a concentration of from about 0.001% to about 0.5% (wt/vol). In preferred embodiments of this aspect of the invention, the surfactant is present in the formulation (prior to lyophilization) in an amount from about 0.005% to about 0.4%; in more preferred embodiments, the surfactant is present in an amount from about 0.01% to about 0.3%. In particularly preferred embodiments, the surfactant is present in an amount from about 0.015% to about 0.1%. In alternate preferred embodiments, the surfactant is present in an amount from about 0.05% to about 0.1%.

In one embodiment of this aspect of the invention, a lyosphere composition comprises (i) antibody; (ii) 12.5% sucrose and 12.5% trehalose; (iii) 10 mM histidine, pH 6.0, and (v) about 0.05% PS-80.

In one embodiment of this aspect of the invention, a lyosphere composition comprises (i) antibody; (ii) 16% sucrose and 5% trehalose; (iii) 10 mM histidine, pH 6.0, and (v) about 0.05% PS-80.

In one embodiment of this aspect of the invention, a lyosphere composition comprises (i) antibody; (ii) 16% sucrose and 5% lactose; (iii) 10 mM histidine, pH 6.0, and (v) about 0.05% PS-80; or

In one embodiment of this aspect of the invention, a lyosphere composition comprises (i) antibody; (ii) 10% raffinose; (iii) 10 mM histidine, pH 6.0, and (v) about 0.05% PS-80.

In certain embodiments of the invention, the stable lyophilized formulations can be reconstituted in 30 minutes or less. In certain embodiments, the lyophilized formulations can be reconstituted in 15 minutes or less, 10 minutes or less, or 5 minutes or less.

Additional components that may be included as appropriate include pharmaceutically acceptable excipients, additives, surface modifiers such as hydroxypropylmethylcellulose (HPMC), diluents, buffers, sugars, amino acids (such as arginine, asparagine, glutamine, glycine, leucine, lysine and proline), chelating agents, surfactants, polyols, bulking agents, stabilizers, cryoprotectants, lyoprotectants, solubilizers, emulsifiers, salts, adjuvants, tonicity enhancing agents (such as alkali metal halides, preferably sodium or potassium chloride, mannitol, sorbitol), delivery vehicles and anti-microbial preservatives. In one embodiment, the lyosphere further comprises an amino acid selected from arginine, proline, lysine, and leucine. In one embodiment, the lyosphere contains 2.5% HPMC.

Other additives might include dyes to distinguish different bead types. Dyes might be visible to the naked eye such as but not limited to food grade dyes, or dyes might include fluorescent dyes and similar dyes that might require a specialized instrument for detection. Food grade dyes are available from, for example, Chromatech, Inc. (Canton, Mich.).

The lysopheres of the present invention are particularly useful for preparing dried pellets from liquid formulations having a low concentration of a therapeutic antibody, e.g. 1 mg/ml or less, and that have a reconstitution time of less than 3 minutes, preferably less than 2 min. The dried pellet is typically stable for at least 1 month at room temperature (e.g., 25° C.), and preferably at least 6 months at 37° C. or at room temperature (e.g., 25° C.). Upon reconstitution, the formulation is suitable for use in a diagnostic analytical assay.

The dried pellets prepared by the method of the present invention can be easily integrated into a variety of reagent amounts by choosing the volume of the droplet used to prepare each pellet. Also, the invention readily enables the preparation of combination products, in which dried pellets comprising one antibody are combined in a single container with dried pellets comprising a different antibody. For example, pellets prepared from antibodies having specificity for different serotypes may be combined in a single container for analytical assays. This may be particularly useful when the antibodies for different stages of the assay have different colors.

The lyospheres can even be stored without stability problems at refrigerator temperature (4-12° C.) or even at room temperature (18-23° C.) over a time period of at least three months, preferably at least six months and in particular of at least one to two years. Furthermore they are also stable when stored at higher temperatures (for example up to 30° C.). The storage stability is for example exhibited by the fact that during the said storage period only a very small number of particles can be detected when the lyospheres are reconstituted in the containers with water. In particular the containers have fewer than 6000 particles with a particle size of more than 10 μm and/or less than 600 particles with a particle size of more than 25 μm.

The fact that the preparations protect against freezing due to the selected combination of additives is particularly advantageous. Hence, in particular this enables a lyophilization at temperatures down to −45° C. without impairing the stability of the antibodies. In addition the lyophilisates containing the combination of additives according to the invention are also stable for a long period and during storage even at relatively high temperatures. Especially compared to conventional formulations, they exhibit no particle formation after reconstitution with water, i.e., the solutions are essentially free of turbidities.

### Kits

The present invention further relates to kits, including kits for use in diagnosis and/or quality control procedures, comprising one or more containers each of which contain lyospheres having an antibody, e.g, a monoclonal antibody (capture, detection or reporter), suitable for an assay step upon reconstitution in diluent, and a container containing an aqueous solution for the reconstitution of the lyosphere(s). A kit may have 1, 2, or 3 containers for antibodies corresponding to the various steps in an immunoassay. For example, for a sandwich ELISA, the kit may contain one container for lyospheres comprising the capture antibody, one container for lyospheres comprising the detection antibody and one container for lyospheres comprising the reporter antibody, as well as one or more containers for reconstitution solutions. For the lyospheres to be suitable for an assay step means that one or more lyospheres can be used for reconstitution.

For kits containing more than one type of antibody, it is preferred that the beads from each container be a different color. Any combination of colors can be used, but it is preferable for the colors to be easily distinguishable by the naked eye. Primary colors such as red, yellow and blue are preferred. White can also be used and can be represented by a clear bead, i.e., a bead with no dye added. Having no dye in a bead containing the capture antibody is preferred in most cases as there is concern the dye may interfere with binding of the capture antibody to the antigen.

In instances where multiple antigens are being assayed, e.g., multiple pneumococcal polysaccharide-protein conjugates, there may be multiple containers each corresponding to a lyospheres comprising an antibody to a conjugate from a particular serotype.

The single-use kits disclosed herein may be produced for various purposes, such as, but not limited to (i) research kits for purposes of clinical studies and other such experimental purposes that may not require the sterile preparation of biological material, (ii) diagnostic kits for use by healthcare practitioners who need to identify the nature and cause of a particular patient ailment, and/or to determine the effectiveness and/or dosage (or other such parameters) of a certain treatment for a particular patient.

The kit may also comprise instructions. The instructions that set forth the method for using each particular type of single-use kit disclosed herein can take a variety of forms or articles. For example, the printed instructions may be a single sheet of paper folded multiple times, an accordion-style folded instruction pamphlet, or a booklet. Instructions may also be presented in various formats, such as quick reference guides and flow charts.

### EXAMPLES

### Example 1: General Procedure for Lyosphere Formation

### Lysophere Formation

In the examples below, frozen droplets of the test compositions were prepared using a metal plate/heat sink apparatus similar to that shown in in International patent Application Publication No. WO2013/066769. The metal plate/heat sink was made of aluminum as large as 10 inches long×7 inches wide×0.4 inches thick and had a flat top surface and a bottom surface with thirty, 1 inch long fins spaced perpendicularly thereto about 0.25 inches apart. The fins were submerged in liquid nitrogen contained in an aluminum reservoir or a styrofoam reservoir that was big enough to hold the metal plate/heat sink.

The beads were formed using a 96-well tip robot that dispenses 50 μL onto a −180° C. flat stainless steel plate. The temperature of the plate insures instantaneous freezing of bead, locking in its semi-sphere shape.

Once formulated the bulk solution containing the antibody and formulation components was transferred to a reservoir from which the 96-well tip robot is programmed to pull up 50 μL per tip and dispense onto the chilled plate. In-between each dispense, from the 96-well head, a hold time is built in to insure beads are fully frozen prior to being shoveled into a collection well. This process of dispensing is repeated until all bulk has been formed into frozen beads.

Alternately, the beads were also formed on a −100° C. stainless steel plate with 4 rows of wells. This method produces spherical beads 100 μL in size. The formulated bulk is dispensed into the wells by 4 dispensing needles. As the needles dispense the previously dispensed beads are shoveled into a collection tray. This process of dispensing is repeated until all bulk has been formed into frozen beads.

Once all dispensing was completed, the frozen beads were transferred from the collection wells to a lyophilizer for drying. The drying of the beads was done using a single step, 30 hour sublimative drying cycle.

### Lyophilization Cycle

Beads were loaded onto a pre-cooled lyo shelf in a metal tray or a plastic tray to facilitate bead drying and handling process. A typical drying cycle is shown below in Table 1.

TABLE 1

Cycle

| | Freezing | Drying |
|---|---|---|
| Temperature (° C.) | −50 | 15 |
| Ramp Rate (° C./min) | 5 | 0.5 |
| Hold Time (min) | 60 | 1800 |
| Vacuum (mTorr) | 30 | 30 |

When drying was complete the beads were unloaded into a low relative humidity (<4% R.H.) glovebox and manually dispensed into glass vials (1 bead per vial).

### Example 2: Feasibility Study

The feasibility of replacing the frozen antibody aliquots currently used as capture and detection reagents in a Total Ps ELISA was evaluated experimentally.

Lyobead formulations and stability were evaluated with reagents that were considered representative of different types of antibody-based immunoassay reagents:
1) Different monoclonal antibodies (mAbs) with different isotypes: IgG1, IgG3, and IgM
2) Different immune-sera/polyclonal antibodies (pAbs)
3) Purified pAb conjugated to alkaline phosphatase

Reagents #1 & #2 were directed against different pneumococcal polysaccharide antigens. Reagent #3 was directed against rabbit antibodies. A total of 7 reagents (3× mAbs, 3× sera, 1× conjugate) were compared as liquid formulation and as lyobead formulation.

Three different ELISAs were carried out against pneumococcal serotypes 1, 9V, and 22F to test the reagents. Each ELISA contained one mAb type, one serum type, and the enzyme conjugate. For example, the ELISA against serotype 1 would feature capture with an IgG1 mAb against pneumococcal polysaccharide of serotype 1, detection with immune-sera specific for the serotype 1 polysaccharide, and the anti-rabbit enzyme conjugated antibody.

Lyobeads were prepared in different buffers as indicated for the individual experiments. Beads (50 μl) contained either 12.5 μg of mAb, immune-sera diluted 10×, or alkaline phosphatase conjugated pAb. For use in the assay, three beads were resuspended in 50 μL of water and then diluted to the appropriate target concentration with Tris-buffered saline containing 1% BSA and 0.05% Tween-20. For example, capture mAbs may have been diluted to a final concentration of 1 μg/mL, or immune sera may have been diluted to a total dilution of 5000×.

Pneumococcal polysaccharides were detected in vaccine product by sandwich ELISA using serotype-specific capture and detection antibodies. Depending on the isotype of the capture antibody, anti-pneumococcal antibodies were either coated directly onto microtiter plates (IgG1 antibodies) or indirectly through capture by an anti-isotype-specific antibody (IgM, IgG3). All steps were separated by three washes with tris-buffered saline+0.05% Tween-20. Immediately prior to substrate addition, plates were washed six times. All steps were carried out at ambient temperature except sample incubation at 2-8° C.

For the indirect assay, a mixture of anti-IgG3- and anti-IgM-specific antibody was coated to plates for 2 hrs at a desired ratio in 1M ammonium sulfate buffer followed by a block step with 1% BSA in tris-buffered saline+0.05% Tween-20 (Diluent T). Then pneumococcus-specific capture antibodies were applied in Diluent

For the direct assay, plates were coated for 2 hrs with pneumococcus-specific capture antibodies diluted in 1M ammonium sulfate followed by a block step in Diluent T.

Both direct and indirect assays were processed similarly downstream of the coating steps with pneumococcal-specific antibodies. Briefly, references and samples were serially diluted in eleven 2-fold steps across the plates and incubated overnight. Starting dilutions were adjusted depending on the serotype of interest. Captured polysaccharides were detected for 1 hr with serotype-specific antibodies diluted, followed by detection with alkaline-phosphatase conjugated anti-species antibody (1 hr) and 4-MuP (45 min).

Relative potencies of samples versus the reference were determined by parallel line analysis of four-parameter fitted curves.

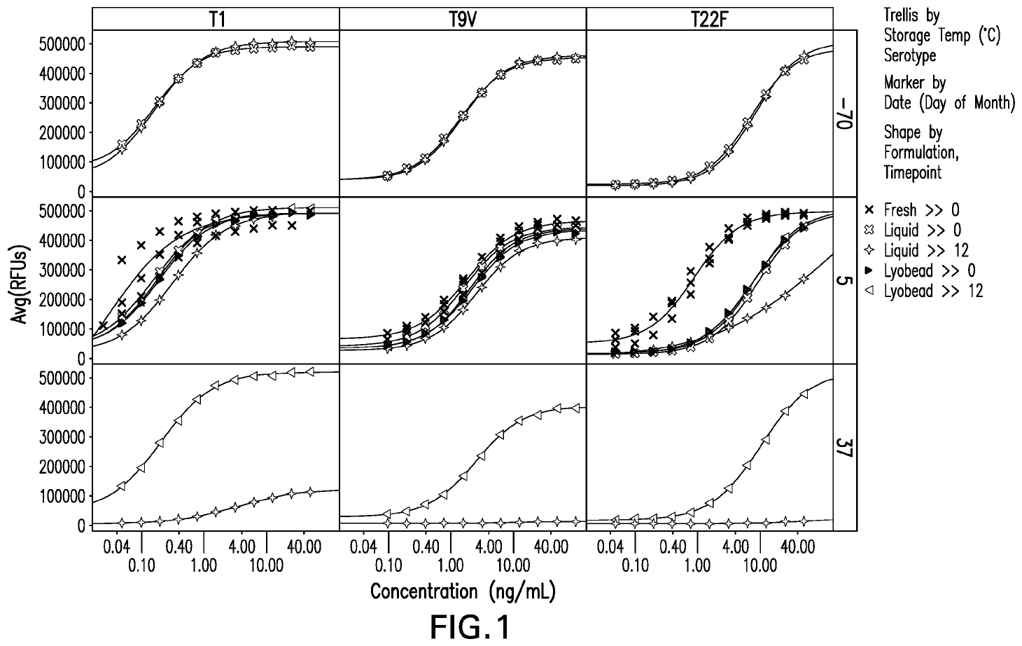

To compare reagent formulations and stability, only reference was applied across the entire plate and curves obtained with different reagents were compared. See FIG. 1.

The data show that the performance of the antibodies when stored as a frozen liquid or a lyosphere is comparable. In the feasibility study, the analysts reported several additional advantages of the lyobeads. Most notably, the analysts reported that the use of the lyobead reagents greatly simplified the assay execution, reducing the opportunity for analyst error. When liquid antibody preparations are used, a different dilution scheme is required for each of the 30 antibody reagents. In contrast, the lyobeads were pre-diluted to an appropriate concentration during formulation. Any variation in the reagent concentration required for testing was accounted for at the time of lyobead preparation. On the day of testing, the analyst was able to apply a standardized reagent preparation process regardless of which reagent was being used. Because all reagents are handled in the same manner, the opportunity for analyst error is greatly reduced. Additionally, the analyst was able to prepare exactly the volume of reagent required for each assay, thereby reducing waste.

Lyobeads were formulated in 12.5% Sucrose+12.5% Trehalose in the base formulation (10 mM His, 0.05% PS-80 pH 6.0). A control arm was not lyophilized and kept at a liquid formulation with the same volume under the same storage conditions. Both arms were subjected to extended storage at −70° C., 5° C., and 37° C.

At chosen intervals, individual tubes were removed from their respective storage condition and tested by ELISA. A control (“fresh”) was included that was prepared using reagents stored in regular conditions. Each plate replicate used a dedicated reagent preparation and lyobead/liquid formulation tube.

Fluorescent signals were plotted against the sample concentration to obtain concentration-response curves. Curves were compared between conditions and across stability time points.

The results are shown in FIG. 1.

Lyobead formulations were found to be stable at least up to 12 months under all storage conditions when compared against the t=0 curve. In contrast, the liquid formulation exhibited degradation at 37° C. as early as 3 months for the serotype 1 assay.

A curve shift was observed compared to the “fresh” control for both lyobeads and liquid formulation. The shift is likely an artifact of the dilution and preparation process.

The 24 month time point also exhibited a curve shift. The shift may be related to a general assay drift rather than the lyobead formulation. However, this was not investigated further and the stability so far was limited to 12 months instead.

### Example 3: 16% Trehalose+5% Sucrose or Lactose Lyobeads

Lyobeads were formulated in either 16% Trehalose+5% Sucrose or in 16% Trehalose+5% Lactose in the base formulation (10 mM His, 0.05% PS-80 pH 6.0). Lyobeads were prepared from 30 different antibody reagents (mAb or immune-sera) and directed against different pneumococcal polysaccharides:
T1, 3, 4, 6A, 6B, 14, 19F, 23F, 33F: IgG1 (direct capture ELISA)
T5, 9V: IgG3 (indirect capture ELISA)
T7F, 18C, 19A, 22F: IgM (indirect capture ELISA)

Concentration response curves with lyobead reagents stored for different times at 37° C. were compared to the t=0 curves and to a “fresh” control.

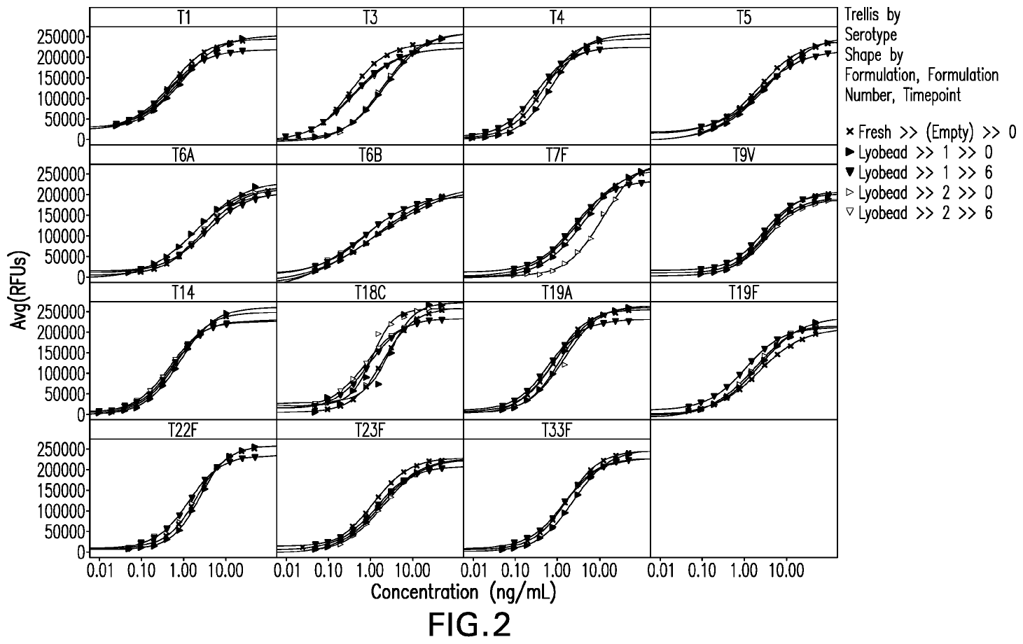

Results are shown in FIG. 2.

No difference was found between the two tested formulations up to 6 months. Both formulations provided reagent stability when taking assay variability into account.

Curve shifts were observed for some conditions in individual serotypes. As no trends were observed, it was concluded that the shifts were likely due to technical error linked to the complexity of the experiments.

### Example 4: 10% Raffinose Lyobeads

Lyobeads were formulated in 10% Raffinose in the base formulation (10 mM His, 0.05% PS-80 pH 6.0) as an alternative buffer applicable to mAbs, sera, and enzyme conjugated antibodies.

Reagents representative of the three classes outlined above, i.e. mAb, immune-sera, and enzyme conjugated antibody. Several serotypes were tested:
T1, 6A: IgG1 mAb/Sera/AP-Conj.
T9V: IgG3 mAb/Sera/AP-Conj.
T7F, 18C, 22F: IgM mAb/Sera/AP-Conj.

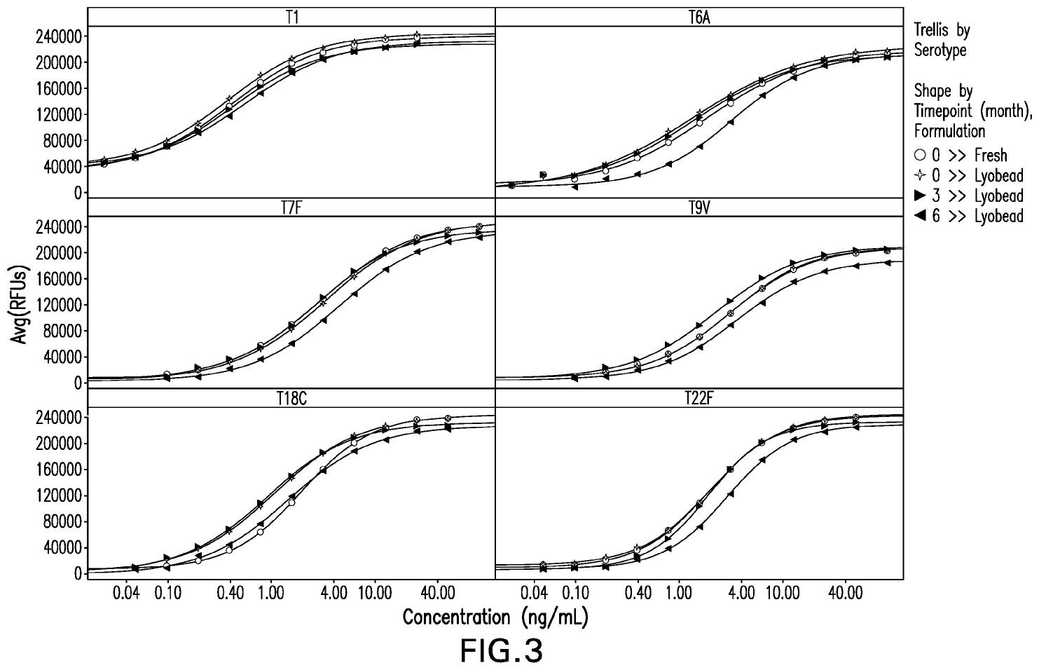

Lyobeads were stored at 37° C. and concentration-response curves from each time points were compared against the curve at t=0 and against a “fresh” control. See FIG. 3.

Lyobead reagents were stable up to 3 months at 37° C. Some curve shifts were observed at 6 months for some serotypes. The assays were still fit-for-purpose at the 6 month time point despite the curve shift.

The Raffinose buffer provided adequate stability to all tested reagents. While Sucrose buffer may provide improved stability, the Raffinose buffer could be used if a single formulation for different reagents is needed.

TABLE 2
Summary of Antibody and Formulation specifications

| | | | | | Other | |
|---|---|---|---|---|---|---|
| | | | | | excipient | |
| | | | | | (even if | |
| | | | | | in small | Ratio of |
| | | mAb/ | AP conjugate | Sugars | quantity | mAb/sugar |
| Exp't | mAb Type | bead | conc. | (w/v) | and pH) | (w/w) |
| 1 | IgG1: 1 | 12.5 μg | 1:10 | 125 mg/mL Sucrose + | 10 mM | 1:10000 |
| | IgG3: 9V | | | 125 mg/mL Trehalose | Histidine, | 1:10000 |
| | IgGM: 22F | | | | 0.5 mg/mL | |
| | | | | | PS80, | |
| | | | | | pH 6.0 | |
| 2 | IgG1: 1 | 12.5 μg | N/A | 100 mg/mL Sucrose | 25 mg/mL | 1:25000 |
| | IgGM: 22F | | | 100 mg/mL Trehalose | HPMC, | 1:25000 |
| | | | | 100 mg/mL Raffinose | 10 mM | 1:25000 |
| | | | | 200 mg/mL Sucrose | Histidine, | 1:12500 |
| | | | | 200 mg/mL Trehalose | 0.5 mg/mL | 1:12500 |
| | | | | 75 mg/mL Sucrose | PS80, | 1:3000 |
| | | | | 75 mg/mL Trehalose | pH 6.0 | 1:3000 |
| | | | | 75 mg/mL Raffinose | | 1:3000 |
| | | | | 125 mg/mL Trehalose + | | 1:10000 |
| | | | | 75 mg/mL Lactose | | 1:3000 |
| 3 | IgG1: 1, 3, | 12.5 μg | 1:10 | 75 mg/mL Raffinose | 25 mg/mL | 1:3000 |
| | 4, 6A, 6B, | | | | HPMC, | |
| | 14, 19F, | | | | 10 mM | |
| | 23F, 33F | | | | Histidine, | |
| | IgG3: 5, 9V | | | | 0.5 mg/mL | |
| | IgM: 7F, | | | | PS80, | |
| | 18C, 19A, 22F | | | | pH 6.0 | |
| 4 | IgG1: 1, 4, | 12.5 μg | 1:10 | 160 mg/mL Trehalose + | 50 mg/mL | 1:15625 |
| | 6A, 23F | | | 50 mg/mL Sucrose | HPMC, | 1:5000 |
| | IgG3: 9V | | | 160 mg/mL Trehalose + | 10 mM | 1:15625 |
| | IgM: 22F | | | 50 mg/mL Lactose | Histidine, | 1:5000 |
| | | | | 160 mg/mL Trehalose | 0.5 mg/mL | |
| | | | | | PS80, pH | |
| | | | | | 5.0, 6.0, | |
| | | | | | and 6.5 | |
| 5 | IgG1: 1, 3, | 12.5 μg | 1:10 | 160 mg/mL Trehalose + | 10 mM | 1:15625 |
| | 4, 6A, 6B, | | | 50 mg/mL Sucrose | Histidine, | 1:5000 |
| | 14, 19F, | | | 160 mg/mL Trehalose + | 0.5 mg/mL | 1:15625 |
| | 23F, 33F | | | 50 mg/mL Lactose | PS80, | 1:5000 |
| | IgG3: 5, 9V | | | | pH 6.0 | |
| | IgM: 7F, | | | | | |
| | 18C, 19A, 22F | | | | | |
| 6 | IgG1: 1, 6A | 12.5 μg | N/A | 100 mg/mL Raffinose | 10 mM | 1:25000 |
| | IgG3: 9V | | | | Histidine, | |
| | IgM: 7F, | | | | 0.5 mg/mL | |
| | 18C, 22F | | | | PS80, | |
| | | | | | pH 6.0 | |
| 7 | IgG1 | 15 μg | 1:5.556 | 100 mg/mL Raffinose | 50 mM | 1:30000 |
| | | | | | Histidine, | |
| | | | | | 50 mM | |
| | | | | | Arginine, | |
| | | | | | 0.5 mg/mL | |
| | | | | | PS80, | |
| | | | | | pH 6.0 | |

1. A lyosphere comprising:
a) an antibody in an amount from about 3 μg to about 45 μg;
b) a buffer to provide a pH from 6.0-8.0;
c) a surfactant; and
d) one or more sugars.

2. The lyosphere of claim 1, wherein the buffer is histidine, succinate, phosphate, MOPS, or any combination thereof.

3. The lyosphere of claim 1, wherein the surfactant is a polysorbate or poloxamer.

4. The lyosphere of claim 1, wherein the surfactant is polysorbate 80, polysorbate 20 or poloxamer P188.

5. The lyosphere of claim 1, wherein the surfactant is at a concentration from about 0.015% to 0.1%.

6. The lyosphere of claim 1, wherein the sugars are dextrose, sucrose, trehalose, lactose, raffinose, or any combination thereof.

7. The lyosphere of claim 1, wherein the sugar is at a concentration of 7.5% or more.

8. The lyosphere of claim 1, wherein the concentration of antibody is in a range from 300 μg/ml to 900 μg/ml.

9. The lyosphere of claim 1, wherein the ratio of mAb/Sugar is in range from 1:3000 to 1:30000 (w/w).

10. The lyosphere of claim 1, wherein the lyosphere is a substantially spherical discrete dried bead having a volume from about 20 μl to about 100 μl.

11. The lyosphere of claim 1, further comprising an amino acid selected from arginine, proline, lysine, and leucine.

12. The lyosphere of claim 1, further comprising hydropropylmethylcellulose (HPMC).

13. The lyosphere of claim 1, wherein the antibody is an IgG1, IgG3, or IgM antibody.

14. The lyosphere of claim 1, wherein the lyosphere does not contain BSA.

15. The lyosphere of claim 1, wherein the antibody is a capture antibody directed to
a) a capsular polysaccharide from a S. pneumoniae serotype;
b) a S. pneumoniae protein selected from PspA, PsaA, PhtA, PhtB, PhtD, or detoxified pneumolysin; or
c) a carrier protein selected from CRM₁₉₇, diphtheria toxin, tetanus toxin, H. influenza protein D, and N. meningitides Outer Membrane Protein Complex (OMPC).

16. The lyosphere of claim 1, wherein the antibody is a detection antibody directed to
a) a capsular polysaccharide from a S. pneumoniae serotype;
b) a S. pneumoniae protein selected from PspA, PsaA, PhtA, PhtB, PhtD, or detoxified pneumolysin; or
c) a carrier protein selected from CRM₁₉₇, diphtheria toxin, tetanus toxin, H. influenza protein D, and N. meningitides Outer Membrane Protein Complex (OMPC).

17. The lyosphere of claim 1, wherein the antibody is a reporter antibody selected from an alkaline phosphatase conjugated antibody (AP-conjugate) or a horse radish peroxidase conjugated antibody (HRP-conjugate).

18. The lyosphere of claim 1, further comprising a colored dye.

19. The lyosphere of claim 1, wherein the lyosphere has a reconstitution time of less than 3 minutes or less than 2 minutes or less than 1 minute.

20. A kit comprising two or more lyospheres according to claim 1, wherein each of the lyospheres comprise a capture antibody, a detection antibody, and/or a reporter antibody.

21. The kit of claim 20, wherein each of the lyospheres comprising the capture antibody, the lyospheres comprising the detection antibody and the lyospheres comprising the reporter antibody are of a different color.

22. The kit of claim 21, wherein each of the lyospheres comprising the capture antibody, the lyospheres comprising the detection antibody and the lyospheres comprising the reporter antibody contain a colored dye.

23. The kit of claim 21, wherein one of the lyospheres comprising the capture antibody, the lyospheres comprising the detection antibody and the lyospheres comprising the reporter antibody does not contain a dye and the other two lyospheres contain a dye.

24. The kit of claim 20, further comprising one or more containers containing a reconstitution liquid.
